(19) United States
(12) Patent Application Publication
(10) Pub. No.: US 2009/0137966 A1
(43) Pub. Date: May 28, 2009

(54) SYRINGE

(75) Inventors: Ralf Rueckert, Wurmlingen (DE); Stefan Knefel, Steisslingen (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: Henke-Sass, Wolf GmbH, Tuttlingen (DE)

(21) Appl. No.: 12/276,086

(22) Filed: Nov. 21, 2008

(30) Foreign Application Priority Data
Nov. 22, 2007 (DE) 102007056240.5

(51) Int. Cl. A61M 5/315

(52) U.S. Cl. 604/227

(57) ABSTRACT

A syringe is provided, comprising a syringe body, a plunger rod, which is guided in the syringe body in a longitudinally displaceable manner, and a grip area which includes a finger portion formed on the syringe body and a thumb portion formed at the rear end of the plunger rod, the grip area being at least partially provided with a friction coating which consists of a different material than the grip area.

### PRIORITY APPLICATION

The current application claims the benefit of priority to German Patent Application No. DE 10 2007 056 240.5 filed on Nov. 22, 2007. Said application is incorporated in its entirety by reference herein.

### BACKGROUND OF THE INVENTION

1. Field of the Invention

The present invention relates to a syringe comprising a syringe body, a plunger rod, which is guided in the syringe body in a longitudinally displaceable manner, and a grip area which includes a finger portion formed on the syringe body and a thumb portion formed at the rear end of the plunger rod.

2. Background

In many cases, such syringes, if they are re-usable syringes, are made from metal. The metal surface is usually as smooth as possible to allow good sterilization or autoclaving of the syringe. However, this leads to the difficulty that it is relatively easy for the fingers to slip off the finger portion or for the thumb to slip off the thumb portion during operation of the syringe.

### SUMMARY OF THE INVENTION

In view thereof, it is an object of the invention to improve a syringe of the above-mentioned type such that easy and safe operation of the syringe can be ensured.

The object is achieved by a syringe of the above-mentioned type in that at least part of the grip area is provided with a friction coating that consists of a different material than the grip area. This friction coating achieves the advantage that there is greater friction between the finger and/or thumb placed on the friction coating and, thus, slipping can be safely prevented.

In particular, the grip area is at least partially sheathed by the friction coating. This is a particularly easy manner of providing the friction coating in the grip area such that the friction coating itself cannot slip with respect to the grip area. Of course, the entire grip area may be sheathed by the friction coating.

The syringe may be provided as a re-usable syringe, in which case the syringe body and the grip area are made substantially from metal. In particular, it is possible for the syringe body and the grip area to be made completely from metal. Such a re-usable syringe can be used for a very long time.

The friction coating may be made from plastics. In particular, it may consist of silicone. For instance, auto-adhesive silicone may be used.

The material of the friction coating is preferably selected such that the syringe remains autoclavable and can, thus, be sterilized very well.

“Autoclavable” is understood here, in particular, to mean that the syringe can be exposed to saturated water vapor at 120-140° C. for at least several minutes for sterilization, without damaging the syringe and, in particular, the friction coating.

The friction coating is made, in particular, from a material that is softer than the material of the grip area. Thus, slipping is additionally prevented by the fingers or the thumbs slightly sinking in during contact with the friction coating.

The thumb portion may have an annular shape. In particular, it may have a circular ring shape or an oval shape. The circular ring-shape design is preferably selected for male users, whereas the oval design of the thumb portion is preferably provided for female users.

The thickness of the friction coating is preferably constant. Of course, it is also possible to vary the thickness and, thereby, to provide e.g. pre-formed cavities for the finger(s) or the thumb.

The friction coating may be applied onto the grip area, in particular by an injection molding method.

The syringe may be provided, in particular, as a carpule syringe. Carpule syringes are frequently used for local anaesthetics in the field of dental medicine. In this case, it is possible to carry out the so-called aspiration test, which involves slight aspiration (by retracting the plunger rod by means of the thumb portion), after the syringe needle has penetrated into the region to be anaesthesized, to ensure that the local-anaesthetics medicament is injected into the appropriate region.

In the syringe, the friction coating may be colored, in which case the color encodes a parameter of the syringe. In particular, this color coding allows to realize an allocation to a user. It is also possible to encode the area of use of the syringe.

The syringe according to the invention is preferably a medical syringe. In particular, it is a syringe used in human medicine, e.g. in dental medicine. However, it may also be provided as a veterinary syringe.

Further, it is also possible to provide a set of the syringes according to the invention, the syringes of the set preferably differing only in the color of the friction coating. This achieves color coding of the otherwise identical syringes so that an undesired mix-up of the individual syringes of the set can be safely avoided.

It will be appreciated that the aforementioned features and those yet to be explained below can be applied not only in the indicated combinations, but also in other combinations, or alone, without departing from the scope of the present invention.

The invention will be explained in more detail below, by way of example and with reference to the enclosed drawings, which also disclose features essential to the invention and wherein:

### BRIEF DESCRIPTION OF THE FIGURES

FIG. 1 shows a perspective view of a first embodiment of the syringe according to the invention;

FIG. 2 shows a sectional view of the syringe of FIG. 1;

FIG. 3 shows a schematic view of a cylindrical ampoule 13;

FIG. 4 shows a perspective view of a second embodiment of the syringe according to the invention, and

FIG. 5 shows a sectional view of the syringe of FIG. 4.

### DETAILED DESCRIPTION

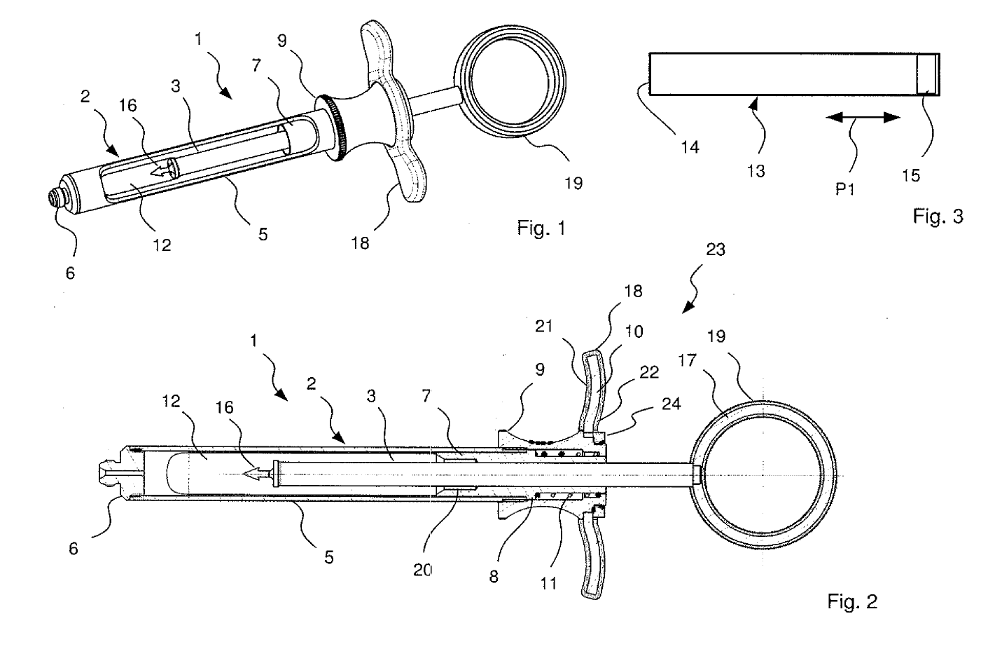

In the embodiments shown in FIGS. 1 and 2, the syringe 1 according to the invention is provided as a carpule syringe, which may also be referred to as a cylindrical-ampoule syringe. The syringe 1 comprises a substantially tubular syringe body 2, in which a plunger rod 3 is supported in a longitudinally displaceable manner.

The syringe body 2 comprises a hollow-cylinder main tube 5 comprising a needle port 6 at the front end and a centering sleeve 7 inserted at the rear end. The needle port 6 is screwed into and glued to the main tube 5. The centering sleeve 7 is inserted into the main tube 5 as far as the stop 8 of the centering sleeve 7, which stop 8 abuts against the rear end of the main tube 5. Further, the rear end of the main tube 5 has a grip cap 9 screwed on and glued to it, and a finger support 10 is rotatably mounted on the rear end of the grip cap 9 and secured by a ring 24. For this purpose, the ring 24 is screwed on and glued to the rear end of the finger support 10.

A spiral spring 11 is arranged between the centering sleeve 7 and the grip cap 9, the ends of said spiral spring 11 being connected to the centering sleeve 7 and the grip cap 9 such that the centering sleeve 7 is pressed against the main tube 5.

The main tube 5 comprises a lateral charging opening 12 via which the syringe 1 can be charged with a cylindrical ampoule 13 schematically shown in FIG. 3. The cylindrical ampoule 13 may be a conventional glass ampoule which is closed at its front end by a membrane layer 14 and at its rear end by a rubber stopper 15 which is displaceable in a longitudinal direction of the cylindrical ampoule 13 (as indicated by the double arrow P1).

The plunger rod 3 has, at its front end, an arrow tip 16 which can be pushed into the cylindrical ampoule 13 inserted in the rubber stopper 15 so that the rubber stopper 15 can be displaced by moving the plunger rod 3 in a longitudinal direction. The arrow tip 16 can thus be stayed in the rubber stopper 15.

A circular ring-shaped pull ring 17 for the thumb of a user of the syringe 1 is mounted to the rear end of the plunger rod 3. The parts of the syringe 1 described so far are all made from metal, with the plunger rod 3 consisting of stainless steel and the remaining parts (except for the compression spring 11) being manufactured from chromed brass.

The syringe body 2 has a length of approximately 110 mm and the inner diameter of the main tube is approximately 10 mm. The plunger rod 3 has a length of approximately 100 mm. The outer and inner diameter of the pull ring 17 in FIG. 2 is approximately 32 or 30 mm, respectively.

In order to facilitate the use of the syringe 1 for an operator, both the finger support 10 (also referred to in the following as finger portion) and the pull ring 17 (also referred to as thumb portion 17) are sheathed by a coating 18 or 19, respectively, of self-adhering silicone. This has the advantage that a higher friction is present between the fingers of the user and the finger portion 10 as well as the thumb of the user and the thumb portion 17 compared with the case in which no silicone sheath 18, 19 is provided and, thus, the fingers are in direct contact with the finger portion 10 and the thumbs are in direct contact with the thumb portion 17. This increased friction facilitates handling of the syringe 1. The thickness of the layers 18 and 19 is preferably as constant as possible. In the exemplary embodiment described herein, it is within a range of from 0.5-1 mm.

In operation, the plunger rod 3 is pulled back by means of the thumb portion 17 until the arrow tip 16 is completely sunk into a receiving region 20 at the front end of the centering sleeve 7. Further retraction of the plunger rod 3 will then cause the centering sleeve 7 to be displaced against the spring 11 in a longitudinal direction of the main tube 5 so that sufficient space is available in the main tube to insert the cylindrical ampoule 13. The plunger rod 3 is held in this position during insertion of the cylindrical ampoule 13. As soon as the cylindrical ampoule 13 has been inserted into the main tube 5 via the lateral charging opening 2, maintenance of the position of the plunger rod 3 can be terminated so that, due to the elastic force of the spring 11, the centering sleeve 7 presses the inserted cylindrical ampoule 13 against the needle port 6 and, thus, fixes the cylindrical ampoule 13 within the main tube 5.

The plunger rod 3 is then pushed forward so as to have the arrow tip 16 pierce the rubber stopper 15. Further, a needle (not shown) is made to pierce the cylindrical ampoule 13 through the needle adaptor 6 and the membrane 14. The syringe 1 is now ready to use. The sheats 18 and 19 prevent the finger from slipping off the finger portion 10 (off its front or rear end 21, 22, depending on how the plunger rod 3 is being moved) as well as preventing the thumb from slipping out of the thumb portion 17.

The finger support 10 and the pull ring 17 form a grip area 23 of the syringe 1. The grip area 23 is designed such that the syringe 1, with a cylindrical ampoule 13 inserted, can be operated single-handedly. In particular, single-hand operation allows the plunger rod 3 to be moved towards the front end of the main tube 5 and away from this front end in a longitudinal direction of the main tube 5.

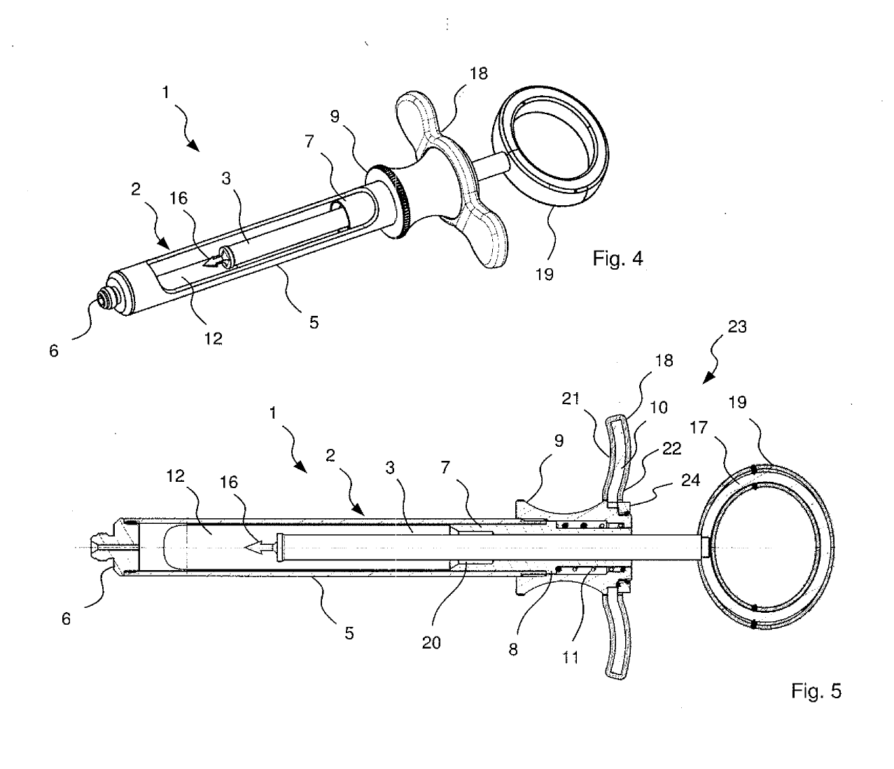

FIGS. 4 and 5 show a modification of the syringe 1 of FIGS. 1 and 2. Like elements are referred to by the same reference numerals, and for description thereof, reference is made to the above statements.

The syringe 1 according to FIGS. 4 and 5 differs from the syringe 1 shown in FIGS. 1 and 2 by the provision of the thumb portion 17. In the syringe of FIGS. 4 and 5, the thumb portion 17 has an oval shape and not a circular ring shape anymore. Further, the length of the plunger rod 3 is slightly reduced. However, the oval thumb portion 17 is also sheathed by a silicone layer 19.

The sheaths 18 and 19 may have a smooth surface. However, it is also possible to select a different surface structure. In particular, the sheaths 18 and 19 are made from a softer material than that from which the finger portion 10 or the thumb portion 17, respectively, is made.

Several syringes 1 of the same design may be provided, differing only in the colors of the sheaths 18 and 19. This achieves the additional advantage that the individual syringes are easy to distinguish, allowing, for example, to easily assign a syringe to a specific operator.

1. A syringe comprising:
a syringe body,
a plunger rod, which is guided in the syringe body in a longitudinally displaceable manner,
and a grip area which includes a finger portion formed on the syringe body and a thumb portion formed at the rear end of the plunger rod,
wherein
the grip area is at least partially provided with a friction coating consisting of a different material than the grip area.

2. The syringe as claimed in claim 1, wherein the grip area is at least partially sheathed by the friction coating.

3. The syringe as claimed in claim 1, wherein the syringe is provided as a re-usable syringe, the syringe body and the grip area being substantially made from metal.

4. The syringe as claimed in claim 1, wherein the friction coating is made from plastic.

5. The syringe as claimed in claim 1, wherein the friction coating is made from silicone.

6. The syringe as claimed in claim 1, wherein the friction coating is softer than a remainder of the grip area.

7. The syringe as claimed in claim 1, wherein the thumb portion has an annular shape.

8. The syringe as claimed in claim 1, wherein the thumb portion has a circular ring shape or an oval shape.

9. The syringe as claimed in claim 1, wherein the friction coating has a constant thickness.

10. The syringe as claimed in claim 1, wherein the syringe is provided as a carpule syringe.

11. The syringe as claimed in claim 1, wherein the friction coating is colored, the color encoding a parameter of the syringe.

12. A set of syringes as claimed in claim 11, wherein the syringes only differ in the color of the friction coating.
